(19) United States
(12) Patent Application Publication
(10) Pub. No.: US 2019/0125568 A1
(43) Pub. Date: May 2, 2019

(54) SHOULDER INJURY ALERT DEVICE

(71) Applicant: Daniel Robert Orcutt, Jonesboro, GA (US)

(72) Inventor: Daniel Robert Orcutt, Jonesboro, GA (US)

(21) Appl. No.: 16/176,502

(22) Filed: Oct. 31, 2018

Related U.S. Application Data
(60) Provisional application No. 62/579,214, filed on Oct. 31, 2017.

(51) Int. Cl. A61F 5/37

(52) U.S. Cl. CPC A61F 5/3723

(57) ABSTRACT

A shoulder injury alert device includes a cover to overlap an anterior and a posterior surface of a wearer's shoulder. The cover may include warning indicia thereon to alert others that the wearer has a shoulder injury. A neck strap is provided to secure a top end of the cover about the patient's shoulder. An arm strap is provided to secure a bottom end of the cover about the patient's upper arm. The straps may be secured with cooperating fasteners. The shoulder injury alert device is intended to alert others of the patient's shoulder condition, which may not be otherwise apparent, particularly when approaching the patient from behind.

### CROSS-REFERENCE TO RELATED APPLICATION

This application claims the benefit of priority of U.S. provisional application No. 62/579,214, filed Oct. 31, 2017, the contents of which are herein incorporated by reference.

### BACKGROUND OF THE INVENTION

The present invention relates to pre and post operative covers, and more particularly, to a shoulder covering for preventing unwanted touching.

Often, patients are accidentally or purposefully touched or hit on the shoulder after an injury or an operative/medical shoulder procedure. Existing devices support the shoulder in a sling, however these devices do not make others realize that the injured area is the shoulder.

As can be seen, there is a need for an apparatus and method for providing a shoulder protective device that covers the shoulder and has a warning symbol for others to refrain from touching the wearer's shoulder.

### SUMMARY OF THE INVENTION

In one aspect of the present invention, a protective shoulder covering to alert others that a patient has a shoulder injury is disclosed. The protective shoulder covering includes a fabric cover dimensioned to overlap a wearer's shoulder covering an anterior and a posterior aspect thereof. A neck strap extends from an upper end of the fabric cover. The neck strap is dimensioned to extend around the wearer's neck and across the wearer's upper torso. An arm strap extends from a lower end of the fabric cover. The arm strap is dimensioned to encircle an upper end of the wearer's arm.

In some embodiments, the protective shoulder covering comprising has a cooperating fastener disposed at a distal end of the neck strap. A cooperating fastener may also be disposed at a distal end of the arm strap. Preferably, the cooperating fastener is a hook and pile fastener.

The protective shoulder covering may be formed of a brightly colored fabric material. A warning symbol may be carried on at least on of a front and back side of the cover.

In other aspects of the invention the protective shoulder covering may include a fabric cover dimensioned to overlap a wearer's shoulder covering the wearer's clavicle with an anterior flap and the wearer's scapula with a posterior flap thereof. A neck strap extends from an upper end of the fabric cover. The neck strap is dimensioned to extend around the wearer's neck and across the wearer's upper torso. An arm strap extends from a lower end of the fabric cover. The arm strap dimensioned to encircle an upper end of the wearer's arm. Warning indicia ma be carried on at least on of a front and back side of the cover to alert others to the patient's injury. A cooperating fastener is disposed at a distal end of the neck strap. Likewise, a cooperating fastener may be disposed at a distal end of the arm strap.

These and other features, aspects and advantages of the present invention will become better understood with reference to the following drawings, description and claims.

### BRIEF DESCRIPTION OF THE DRAWINGS

FIG. 1 is a perspective view of the shoulder injury alert device, shown in use;

FIG. 2 is a top front perspective view of the shoulder injury alert device, with straps 14 and 16 open;

FIG. 3 is a top rear perspective view of the shoulder injury alert device, with straps 14 and 16 closed and straps 22 and 24 open; and

FIG. 4 is a perspective view of the shoulder injury alert device laid flat.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

As previously stated, patients often struggle with accidental or intentional touching of the shoulder after a medical procedure. The invention claimed here solves this problem.

Broadly, embodiments of the present invention provide a covering for the shoulder that makes people aware that the wearer has a painful shoulder. The cover includes a warning symbol or indicia on it in order to make others aware that there is something wrong with the wearer's shoulder.

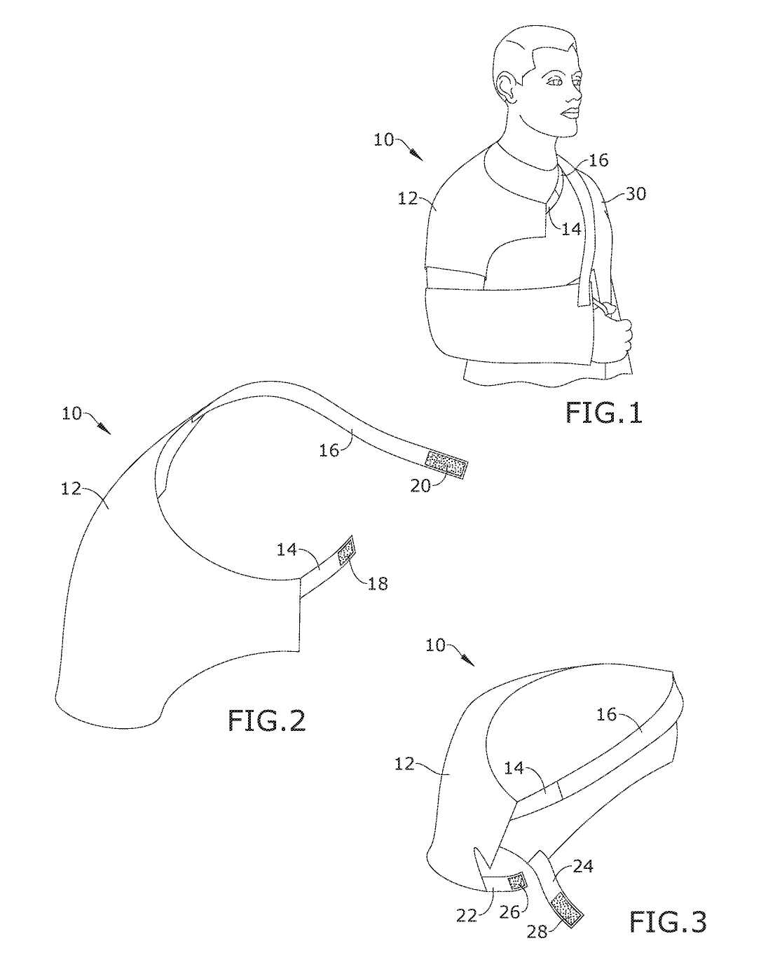

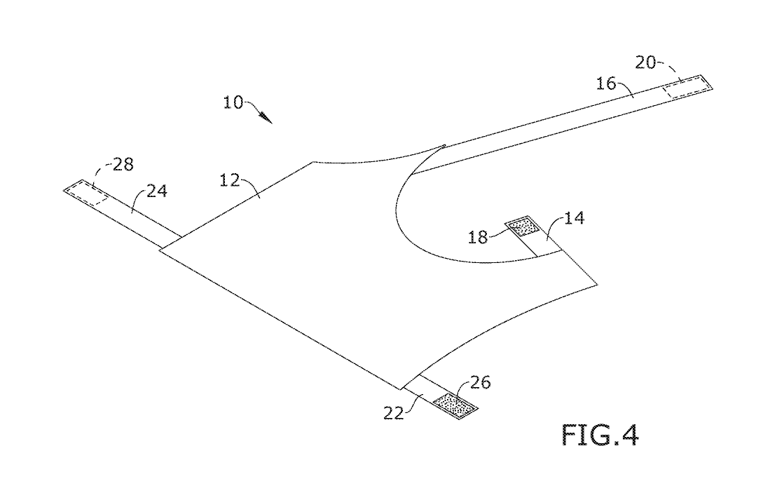

As shown in the embodiments of FIGS. 1 through 4, embodiments of the shoulder injury alert device 10 of present invention includes a covering 12 for the to be worn by a patient 30 with a shoulder injury. The covering 12 is secured to the shoulder, via a neck strap 14, 16 and a cooperating fastener 18, 20, such as Velcro®, The cooperating fastener 18, 20 may also include a button, a snap, a hook and loop, and the like. The neck strap 14, 16 is of sufficient length to extend around the wearer's neck so that the straps extend about the anterior thorax. The cooperating fasteners 18, 20 are positioned so that the wearer may adjust the strap 14, 16 with their good hand.

In some embodiments, the covering 12 is placed over the shoulder and then secured to the upper arm of the injured shoulder via an arm strap 22, 24 with a cooperating fastener 26, 28 such as the Velcro® depicted. The cooperating fastener may also include a button, a snap, a hook and loop, and the like. The shoulder covering 12 is secured to the posterior thorax through a third strap that clips onto the back of the shirt. Alternatively, the two thorax straps can be connected around the thorax.

The cover 12 material is a type of cotton fabric blend. It is to be understood, however, that the material can be any suitable type of fabric. In one embodiment, the shoulder covering 12 is a brightly colored material. The cover 12 may also be stamped with a warning symbol (either a stop sign, a white cross, or other alerting indicia) on the back and front so that it is visible when approaching the patient from the front and from behind so as to alert others to the patient's injury. A front covering surface of the cover 12 extends across a clavicle of the wearer 30. A back surface of the cover 12 may extend over the scapula of the wearer 30.

To make the shoulder injury alert device 10, a piece of brightly colored cloth is sewn in such a way that it can cover (or cup) the shoulder. The cover 12 may be imprinted on the front and back with a white cross or a stop sign (octagon). Attached to the cover 12 are 4 straps. Two shorter straps 22, 24 are dimensioned to surround the upper arm and connect to each other using cooperating fasteners 26, 28, such as Velcro®. Two longer straps 14, 16 may be connected to the covering 12 with plastic clips that can be attached to a shirt or a bra strap, or may be attached to each other around the body. Bright colored fabric may be imprinted with two emblems one on the back and one on the front of the material. The emblem may be, for example, either a white cross or a stop sign.

In another embodiment, an additional mechanism is used that connects the two more centrally located straps around the thorax.

It is to be understood that the color and the type of warning symbol used can be changed per preference. Also, the straps can be changed or adjusted per the user's comfort.

It should be understood, of course, that the foregoing relates to exemplary embodiments of the invention and that modifications may be made without departing from the spirit and scope of the invention as set forth in the following claims.

What is claimed is:

1. A protective shoulder covering comprising:
a fabric cover dimensioned to overlap a wearer's shoulder covering an anterior and a posterior aspect thereof;
a neck strap extending from an upper end of the fabric cover, the neck strap dimensioned to extend around the wearer's neck and across the wearer's upper torso; and
an arm strap extending from a lower end of the fabric cover, the arm strap dimensioned to encircle an upper end of the wearer's arm.

2. The protective shoulder covering of claim 1, further comprising:
a cooperating fastener disposed at a distal end of the neck strap.

3. The protective shoulder covering of claim 2, further comprising:
a cooperating fastener disposed at a distal end of the arm strap.

4. The protective shoulder covering of claim 3, wherein the cooperating fastener is a hook and pile fastener.

5. The protective shoulder covering of claim 3, wherein the fabric cover further comprises:
a brightly colored fabric material.

6. The protective shoulder covering of claim 3, wherein the fabric cover further comprises:
a warning symbol on at least on of a front and back side of the cover.

7. A protective shoulder covering comprising:
a fabric cover dimensioned to overlap a wearer's shoulder covering the wearer's clavicle with an anterior flap and the wearer's scapula with a posterior flap thereof;
a neck strap extending from an upper end of the fabric cover, the neck strap dimensioned to extend around the wearer's neck and across the wearer's upper torso; and
an arm strap extending from a lower end of the fabric cover, the arm strap dimensioned to encircle an upper end of the wearer's arm.

8. The protective shoulder covering of claim 7, further comprising
a warning indicia carried on at least on of a front and back side of the cover.

9. The protective shoulder covering of claim 8, further comprising:
a cooperating fastener disposed at a distal end of the neck strap.

10. The protective shoulder covering of claim 9, further comprising:
a cooperating fastener disposed at a distal end of the arm strap.
